(19) United States
(12) Patent Application Publication
(10) Pub. No.: US 2024/0090556 A1
(43) Pub. Date: Mar. 21, 2024

(54) Liquid Nutritional Compositions with Water-Insoluble Plant Flavonoid and Method of Production Thereof

(71) Applicant: ABBOTT LABORATORIES, Abbott Park, IL (US)

(72) Inventors: Quang Son PHAM, Powell, OH (US); Suzette L. PEREIRA, Westerville, OH (US); Paul JOHNS, Columbus, OH (US); Susan WANG, Columbus, OH (US); Megan TERP, Columbus, OH (US); Ricardo Rueda Cabrera, Granada (ES)

(21) Appl. No.: 18/255,010

(22) PCT Filed: Nov. 29, 2021
(86) PCT No.: PCT/US21/60921

§ 371 Date: May 30, 2023

Related U.S. Application Data
(60) Provisional application No. 63/119,157, filed on Nov. 30, 2020.

(51) Int. Cl. A23L 33/105; A23L 33/115; A23L 33/15; A23L 33/185; A23L 33/19

(52) U.S. Cl. CPC A23L 33/105; A23L 33/115; A23L 33/15; A23L 33/185; A23L 33/19

(57) ABSTRACT

A method of forming a heat-treated liquid nutritional composition having a neutral pH and comprising a water-insoluble plant flavonoid comprises providing an aqueous liquid nutritional composition having a pH of from about 6 to about 7.5 and comprising protein, fat, carbohydrate, and water-insoluble plant flavonoid, homogenizing the liquid nutritional composition at a pressure of at least about 2000 psi, and heat treating the liquid nutritional composition. A heat-treated liquid nutritional composition having a pH of from about 6 to about 7.5 comprises a water-insoluble plant flavonoid, protein, fat and carbohydrate. At least about 75 wt % of the water-insoluble plant flavonoid remains suspended throughout the liquid nutritional composition after two months of storage at room temperature.

### FIELD OF THE INVENTION

The present invention is directed to liquid nutritional compositions comprising at least one water-insoluble plant flavonoid, and specifically, to such compositions which exhibit improved suspension stability of the water-insoluble plant flavonoid therein, and to methods for preparing such compositions. The invention is particularly advantageous for liquid nutritional compositions having a relatively neutral pH of from about 6 to about 7.5 and which have been heat treated.

### BACKGROUND OF THE INVENTION

Plant flavonoids are found in various parts of plants such as fruits and vegetables, and in tea, cocoa, and wine, and have been studied for many health benefits. Water-insoluble plant flavonoids such as water-insoluble citrus flavonoids are known to have various health benefits, including cardiovascular benefits such as lowering blood pressure and improving blood flow, improving muscle performance in the context of exercise, and improving mitochondrial function leading to improved strength and energy. However, owing to the water insolubility of such flavonoids, incorporating them into beverages which are shelf stable is notoriously difficult, particularly in pH-neutral liquid products and/or heat-treated beverages, due to challenges in achieving a uniform dispersion or suspension. Generally, over time, these water-insoluble flavonoids sediment to the bottom of the beverage container. This makes it difficult, if not impossible, to deliver a consistent dose of the water-insoluble flavonoids in a liquid product, especially in a product needing shelf life stability.

In the past, the water-insoluble citrus flavonoid hesperidin has been complexed with various molecules in attempts to improve its solubility/dispersibility. These include cyclodextrin (US 2013/0309218 A1), 2-hydroxypropyl-beta-cyclodextrin (HP-β-CD) (Majumbdar et al, Pharm Res., 26(5): 1217-1225 (2009)), D-glucose to form α-glycosyl hesperidin (JP 3549436 B2 and JP 2007308414 A), and various polymers (Cao et al, J Sci Food Agric, 98: 2422-2427 (2018)). However, such complexes can change the biological properties of the flavonoid and/or affect its chemical characteristics, and many of these complexed structures may not be suitable for use in beverages intended for daily or otherwise regular consumption.

Another method for improving the solubility/dispersibility of the citrus flavonoid hesperidin comprises dissolving hesperidin in a strong alkali solution, and then adding a polysaccharide thickener to adjust the pH of the solution (JP 4202439 B2). However, it is known that hesperidin is extremely unstable, especially against light and oxygen, in the alkali pH range, and decomposition occurs during storage.

Another attempt to stabilize hesperidin involves combining it with a specific dihydric alcohol, and a sugar alcohol, wherein the decomposition of hesperidin is suppressed and enables hesperidin dissolution/dispersion without the use of a strong alkali solution (U.S. Pat. No. 8,507,452 B2). However, the specific dihydric alcohols and sugar alcohols which are employed are not typically used or acceptable in beverages intended for daily consumption.

Thus, there is a need for a method to stabilize and uniformly disperse water-insoluble bioactive flavonoids such as the citrus flavonoid hesperidin in a beverage for daily consumption, such as in a liquid nutritional product, that exhibits storage stability, and, specifically, that can survive heat processing and remain in suspension over time.

### SUMMARY OF THE INVENTION

Accordingly, it is an object of the invention to provide a method for improving the suspension stability of water-insoluble plant flavonoids in beverages, and, more specifically, in liquid nutritional compositions, including liquid nutritional compositions which have a neutral pH of from about 6 to about 7.5 and which have been heat treated. A related object is to provide improved liquid nutritional compositions have at least one water-insoluble plant flavonoid dispersed therein.

Therefore, in one embodiment, the invention is directed to a method of forming a heat-treated liquid nutritional composition having a neutral pH, i.e., a pH of from about 6 to about 7.5, and comprising a water-insoluble plant flavonoid. The method comprises providing an aqueous liquid nutritional composition having a pH of from about 6 to about 7.5 and comprising protein, fat, carbohydrate, and water-insoluble plant flavonoid, homogenizing the liquid nutritional composition at a pressure of at least 2000 psi, and heat treating the liquid nutritional composition.

In another embodiment, the invention is directed to a heat-treated liquid nutritional composition having a pH of from about 6 to about 7.5 and comprising a water-insoluble plant flavonoid and protein. At least about 75 wt % of the water-insoluble plant flavonoid remains suspended throughout the liquid nutritional composition after two months of storage at room temperature.

The methods and liquid nutritional compositions of the invention are advantageous in allowing the incorporation of water-insoluble plant flavonoids in storage stable liquid nutritional compositions which are acceptable for routine consumption, including daily consumption, and which allow predictable dosing of the water-insoluble plant flavonoids. These and additional aspects and advantages of the invention will be more fully described in the detailed description.

### BRIEF DESCRIPTION OF THE DRAWINGS

The drawings may facilitate understanding of certain aspects or embodiments of the invention, wherein

FIG. 1 shows a relationship of the water-insoluble flavonoid hesperidin and protein, on a weight basis, in, respectively, a conventional liquid nutritional composition and a liquid nutritional composition prepared according to the method of the invention; and

FIG. 2 shows a relationship of the water-insoluble flavonoid hesperidin and protein, on a molar basis, in, respectively, a conventional liquid nutritional composition and a liquid nutritional composition prepared according to the method of the invention.

### DETAILED DESCRIPTION

While the general inventive concepts are susceptible of embodiment in many different forms, described herein in detail are specific embodiments of the invention, with the understanding that the present disclosure is to be considered as an exemplification of the principles of the general inventive concepts. Accordingly, the general inventive concepts are not intended to be limited to the specific embodiments illustrated and described herein.

The invention is directed to liquid nutritional compositions and to methods for preparing liquid nutritional compositions. The term “liquid nutritional composition” as used herein, unless otherwise specified, encompasses all forms of liquid nutritional compositions, including emulsified liquids, concentrated liquids intended for dilution, for example, by addition of water, and ready-to-drink liquids. The nutritional compositions are suitable for oral consumption by a human.

All percentages, parts and ratios as used herein, are by weight of the total composition, unless otherwise specified. All such weights as they pertain to listed ingredients are based on the active level and, therefore, do not include solvents or byproducts that may be included in commercially available materials, unless otherwise specified.

The terminology as set forth herein is for description of the embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

Throughout this specification, when a range of values is defined with respect to a particular characteristic of the present invention, the present invention relates to and explicitly incorporates every specific subrange therein. Additionally, throughout this specification, when a group of substances is defined with respect to a particular characteristic of the present invention, the present invention relates to and explicitly incorporates every specific subgroup therein. Any specified range or group is to be understood as a shorthand way of referring to every member of a range or group individually as well as every possible subrange or subgroup encompassed therein.

The various embodiments of the liquid nutritional compositions employed in the methods of the present disclosure may also be substantially free of any optional or selected ingredient or feature described herein, provided that the remaining nutritional composition still contains all of the required ingredients or features as described herein. In this context, and unless otherwise specified, the term “substantially free” means that the selected nutritional composition contains less than a functional amount of the optional ingredient, typically less than 1%, including less than 0.5%, including less than 0.1%, and also including zero percent, by weight, of such optional or selected essential ingredient.

The methods and nutritional compositions described herein may comprise, consist of, or consist essentially of the essential steps and elements, respectively, as described herein, as well as any additional or optional steps and elements, respectively, described herein. Any combination of method or process steps as used herein may be performed in any order, unless otherwise specified or clearly implied to the contrary by the context in which the referenced combination is made.

Unless otherwise indicated herein, all exemplary embodiments, sub-embodiments, specific embodiments and optional embodiments are respective exemplary embodiments, sub-embodiments, specific embodiments and optional embodiments to all embodiments described herein.

In a first embodiment, the invention is directed to a method of forming a heat-treated liquid nutritional composition having a neutral pH, i.e., a pH of from about 6 to about 7.5, or, in a more specific embodiment, a pH of from about 6.5 to about 7.2, and comprising a water-insoluble plant flavonoid. As discussed, it has been difficult to provide such liquid nutritional compositions with homogeneous and stable suspension of the water-insoluble plant flavonoid. It has been especially difficult to provide good suspension of the water-insoluble plant flavonoid in liquid nutritional compositions which have been heat treated, which is needed in order to provide sterilized, storage stable products. While not wishing to be limited by theory, it is believed that the methods of the invention result in an increased association between the water-insoluble flavonoid and protein in the composition, which association allows the flavonoid to remain in suspension in the composition with protein during heat treatment and storage of the liquid nutritional composition. Advantageously, this method does not change the chemical characteristics of the water-insoluble flavonoid molecule and no new or non-food grade chemicals need to be added to the nutritional composition to achieve a stable suspension of the water-insoluble flavonoid in the liquid nutritional composition. Additionally, and importantly, the flavonoid-protein association appears to be achieved through hydrogen bonding and/or hydrophobic interactions. As the protein is digested upon consumption of the nutritional composition, those bonds are broken and the flavonoid is released to provide beneficial effects.

The methods comprise providing an aqueous liquid nutritional composition having a pH of from about 6 to about 7.5 and comprising protein, fat, carbohydrate, and water-insoluble plant flavonoid, and homogenizing the liquid nutritional composition under a relatively high pressure. As is well known in the art, homogenization may be achieved using any of a variety of commercially available homogenizer equipment, including blenders, bead mills, ultrasonic systems (sonication), rotor-stator devices, and other high pressure devices which provide sufficient physical forces to form an increased association between the protein and flavonoid in the composition. Within the present specification, high pressure homogenization refers to a homogenization process employing a pressure of at least 2000 psi. Pressures ranging from 2000 to about 5000 psi may suitably be employed, in one or several stages. In specific embodiments, the homogenizing step comprises at least one homogenization stage at a pressure of from 2000 to about 5000 psi, or from about 2500 to about 4500 psi, or from about 3000 to about 4500. In additional specific embodiments, the homogenizing step comprises a two stage homogenization, with a first stage at a pressure of at least 2000 psi, or more specifically, from 2000 to about 5000 psi, from about 2500 to about 4500, from about 3000 to about 4500, and a second stage at a lower pressure, for example at a pressure of from about 250 to about 1500 psi, from about 250 to about 1000 psi, or from about 250 to about 750 psi.

The amount of water-insoluble flavonoid which is included in the nutritional composition, may vary depending on a desired dosage of the flavonoid for the nutritional product. In specific embodiments, the final liquid nutritional composition comprises from about 50 to about 800 mg, from about 50 to about 600 mg, or from about 50 to about 300 mg water-insoluble plant flavonoid per 237 ml serving of the liquid nutritional composition. The aqueous liquid nutritional composition desirably contains sufficient protein to increase association of substantially the entire amount of water-insoluble flavonoid with the protein during the homogenization step. In specific embodiments, the aqueous liquid nutritional composition comprises a weight ratio of protein to flavonoid of from about 5 to about 100, from about 10 to about 50, or from about 15 to about 25.

The methods of the present invention, and the resulting compositions, may employ one or more water-insoluble plant flavonoids. In specific embodiments, the water-insoluble plant flavonoid comprises a citrus flavonoid, or, more specifically, the water-insoluble plant flavonoid comprises at least one of hesperidin and hesperetin, the aglycone of hesperidin. These two flavonoids from citrus species have numerous biological properties, particularly antioxidant and anti-inflammatory properties. More specifically, water-insoluble citrus flavonoids such as hesperidin provide cardiovascular benefits, including lowering blood pressure, improving blood flow, improving muscle performance in context of exercise, and improving mitochondrial function leading to improved strength and energy, including improved muscle function and mass. These flavonoids are therefore advantageous in providing such health benefits via the inventive liquid nutritional compositions. In a yet more specific embodiment, the water-insoluble flavonoid comprises hesperidin, alone or in combination with one or more additional water-insoluble flavonoids such as, for example, narirutin, diosmin, naringin, didymin, and/or hesperetin. In more specific embodiments, the nutritional compositions comprise from about 50 to about 500 mg, or from about 100 to about 300 mg, water-insoluble citrus flavonoid, or, more specifically, hesperidin, per a 237 ml serving of the liquid nutritional composition. In additional specific embodiments, the nutritional compositions comprise from about 50 to about 250 mg water-insoluble citrus flavonoid, or, more specifically, hesperidin, per a 237 ml serving of the liquid nutritional composition. In other specific embodiments, the nutritional compositions comprise hesperetin, or, more specifically, comprise from about 50 to about 150 mg hesperetin per a 237 ml serving of the liquid nutritional composition.

In additional embodiments, the water-insoluble plant flavonoid comprises resveratrol, which is found in the skin of grapes, blueberries, raspberries, mulberries, and peanuts, and is known to act as an antioxidant, and therefore is advantageous in providing antioxidant benefits via the inventive liquid nutritional compositions. In more specific embodiments, the nutritional compositions comprise from about 50 to about 500 mg, or from about 100 to about 300 mg, resveratrol per a 237 ml serving of the liquid nutritional composition.

In a specific embodiment, the water-insoluble flavonoid has a human serum albumin (HSA) binding constant that allows effective transport via the human bloodstream but also allows release at target tissue. Therapeutic agent binding to HSA may be characterized by the Stern-Volmer binding constant (K_{b}), where a higher value indicates a higher affinity, or a stronger (non-covalent) bond between the therapeutic agent and HSA. In one embodiment, the flavonoid employed in the methods and compositions herein has a K_{b} in a range of 10⁴-10⁵ M⁻¹ and, more specifically, has a K_{b} in the range of 1×10⁴-5×10⁴ M⁻¹. In addition to hesperidin, hesperetin and resveratrol, water-insoluble flavonoids such as rutin, isovitexin, quercetin, chlorogenic acid, and 2-phenylchromone exhibit K_{b} within these ranges and are suitable for use in this specific embodiment. Any of the water-insoluble flavonoids may be used individually or in combinations of two or more, as desired.

The protein which is contained in the nutritional composition may be any one or more proteins known for use in nutritional compositions. A wide variety of sources and types of protein can be used in the nutritional compositions. For example, the source of protein may include, but is not limited to, intact, hydrolyzed, and partially hydrolyzed protein, which may be derived from any suitable source such as milk (e.g., casein, whey), animal (e.g., meat, fish), cereal (e.g., rice, brown rice, corn, barley, etc.), vegetable (e.g., soy, pea, yellow pea, fava bean, chickpea, canola, potato, mung, ancient grains such as quinoa, amaranth, and chia, hemp, flax seed, etc.), and combinations of two or more thereof. The protein may also include one or a mixture of naturally occurring or synthetic amino acids (often described as free amino acids) and/or their metabolites, known for use in nutritional products, alone or in combination with the intact, hydrolyzed, and/or partially hydrolyzed proteins described herein.

More specific examples of sources of protein which are suitable for use in the exemplary nutritional compositions described herein include, but are not limited to, whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, barley rice protein, fava bean protein concentrate, fava bean protein isolate, fava bean protein hydrolysate, collagen proteins, collagen protein isolates, meat proteins such as beef protein isolate and/or chicken protein isolate, potato proteins, chickpea proteins, canola proteins, mung proteins, quinoa proteins, amaranth proteins, chia proteins, hemp proteins, flax seed proteins, earthworm proteins, insect proteins, and combinations of two or more thereof. The nutritional compositions can include any individual source of protein or combination of any of the various sources of protein listed above. Any one or more of these proteins may be employed in the nutritional compositions.

In a specific embodiment, the nutritional composition contains at least one milk protein, more specifically, milk protein concentrate, milk protein isolate, and/or calcium and/or sodium caseinates. In more specific embodiments, the nutritional composition contains at least one milk protein and at least one plant protein, or, more specifically, at least one soy protein, for example, soy protein isolate. In additional embodiments, the nutritional composition contains at least one plant protein. In more specific embodiments, the protein comprises one or more milk proteins and a soy protein, and more specifically, from about 50 to about 90 wt % milk protein and from about 10 to about 50 wt % soy protein.

The nutritional composition also includes fat and carbohydrate. The term “fat” as used herein, unless otherwise specified, refers to lipids, fats, oils, and combinations thereof. Sources of fat suitable for use in the nutritional composition include, but are not limited to, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, lecithin, and long chain polyunsaturated fatty acids such as docosahexanoic acid (DHA), arachidonic acid (ARA), docosapentaenoic acid (DPA), eicosapentaenoic acid (EPA), and combinations thereof. The nutritional compositions can include any individual source of fat or combination of any of the various sources of fat listed above.

Sources of carbohydrates suitable for use in the nutritional compositions may be simple or complex, or variations, or combinations thereof. Various sources of carbohydrate may be used so long as the source is suitable for use in a nutritional composition and is otherwise compatible with any other selected ingredients or features present in the nutritional composition. Non-limiting examples of sources of carbohydrates suitable for use in the nutritional compositions include maltodextrin, hydrolyzed or modified starch, hydrolyzed or modified cornstarch, glucose polymers such as polydextrose and dextrins, corn syrup, corn syrup solids, rice-derived carbohydrates such as rice maltodextrin, brown rice mild powder and brown rice syrup, sucrose, glucose, fructose, lactose, high fructose corn syrup, honey, sugar alcohols (e.g., maltitol, erythritol, sorbitol), isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, Fibersol™, fruit puree, vegetable puree, isomalto-oligosaccharides, monosaccharides, disaccharides, human milk oligosaccharides (HMOs), tapioca-derived carbohydrates, inulin, other digestion-resistant starches, and artificial sweeteners, and combinations of two or more thereof. The nutritional compositions may include any individual source of carbohydrate or combination of any of the various sources of carbohydrate listed above.

The protein may be combined with fat and carbohydrate in relative amounts necessary to provide a liquid nutritional composition with the desired contents of protein and fat and/or carbohydrate. The concentration and relative amounts of the sources of protein, and of carbohydrate and/or fat in the nutritional compositions can vary considerably depending upon, for example, the specific dietary needs of the intended user. For example, in specific embodiments, the one or more sources of protein comprise from about 1 wt % to about 25 wt % of the nutritional composition. In more specific embodiments, the one or more sources of protein comprise from about 2 wt % to about 20 wt %, about 2 wt % to about 15 wt %, about 5 wt % to about 20 wt %, about 5 wt % to about 25 wt %, or about 5 wt % to about 15 wt % of the nutritional composition. In additional specific embodiments, the liquid nutritional composition comprises protein in an amount of from about 2 to about 20 g, from about 2 to about 15 g, or from about 2 to about 10 g, per 100 ml of the liquid nutritional composition.

In additional specific embodiments, the one or more sources of fat comprise about 0.5 wt % to about 20 wt % of a source of fat. In more specific embodiments, one or more sources of fat comprise about 0.5 wt % to about 18 wt % of the nutritional composition, including about 0.5 wt % to about 15 wt %, about 0.5 wt % to about 10 wt %, about 0.5 wt % to about 5 wt %, about 2 wt % to about 8 wt %, about 2 wt % to about 10 wt %, about 5 wt % to about 15 wt %, or about 5 wt % to about 20 wt % of the nutritional composition. In additional specific embodiments, the liquid nutritional composition comprises fat in an amount of from about 0.5 to about 15 g, from about 0.5 to about 10 g, or from about 1 to about 5 g, per 100 ml of the liquid nutritional composition.

In additional specific embodiments, the one or more sources of carbohydrate are present in an amount from about 5 wt % to about 50 wt % of the nutritional composition. In more specific embodiments, the one or more sources of carbohydrate are present in an amount from about 5 wt % to about 30 wt % of the nutritional composition, about 5 wt % to about 25 wt %, about 5 wt % to about 20 wt %, about 5 wt % to about 15 wt %, about 10 wt % to about 25 wt %, or about 10 wt % to about 20 wt %, of the nutritional composition. In additional specific embodiments, the liquid nutritional composition comprises carbohydrate in an amount of from about 5 to about 25 g, from about 5 to about 20 g, or from about 10 to about 20 g, per 100 ml of the liquid nutritional composition.

The concentration and relative amounts of the sources of protein, carbohydrate, and fat in the liquid nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 1 wt % to about 10 wt %, based on the weight of the nutritional composition.

The components of the liquid nutritional compositions may be combined in any desired manner. In a specific embodiment of one suitable manufacturing process, the nutritional composition may be formed by combining an aqueous protein-in-water (PIW) slurry with a protein-in-fat (PIF) slurry and a carbohydrate-mineral (CHO-MN) slurry. The slurries are blended together with heat and agitation. In a specific embodiment, the blended slurries are then homogenized according to the high pressure homogenization discussed above. While conventional processes may have included an emulsification step in preparing liquid nutritional compositions using a homogenizer, such emulsification is typically done at a lower pressure, for example, less than 1200 psi, or more specifically, at about 900-1100 psi, and not at the high pressure of at least 2000 psi required by the present invention. If desired, the blended slurries may be subjected to such an emulsification step, for example prior to the high pressure homogenization required by the present invention. A pH adjustment may be made as needed during the manufacture, for example at the blend stage when the slurries are blended, or afterheat processing and cooling, or elsewhere in the process, to the desired neutral range, typically from about 6 to about 7.5, or more specifically, from about 6.6 to about 7.2. The water-insoluble flavonoid may be added to any one of the slurries, or to the resulting blend, prior to the high pressure homogenization. Any additional components, for example, water soluble vitamins, flavors and the like may be added to the resulting blend, for example, after cooling, as well. Water may also added to achieve the desired total solids level (typically 15-40 total solids wt %).

The specific method steps for combining the various components to form the nutritional composition may be carried out in various ways other than those described above without departing from the spirit and scope of the present invention.

The nutritional composition is subjected to a heat treatment which provides sterilization sufficient to maintain microbiological stability of the liquid nutritional composition over a desired shelf-life. In a specific embodiment, the heat treatment should be sufficient to maintain stability of the liquid nutritional composition over a shelf-life of at least about 12 months. The specific heating conditions are not critical and various heating processes known in the art may be employed. In one embodiment, the composition may be heat treated, for example by high temperature, short time (HTST) heat treatment, for example at a temperature of from about 165° F. to about 185° F. (from about 70° C. to about 85° C.) for about 16 to 25 seconds. In another embodiment, the composition may be heat treated, for example, by ultra-high temperature (UHT) heat treatment, for example, at a temperature of from about 280° F. to about 305° F. (from about 138° C. to about 152° C.), for at least about 5 seconds. In either such embodiments, the heat treatment may be conducted before, simultaneous with, or after the high pressure homogenization. In more specific embodiments, the composition may be subjected to the high pressure homogenization, followed by HTST heat treatment, and then cooled. In additional more specific embodiments, the heat treatment comprises a UHT processing step followed by the high pressure homogenization.

The thus formed liquid nutritional compositions may then be packaged as desired. In specific embodiments, liquid nutritional compositions are aseptic or retort packaged. In a specific embodiment, an optionally UHT treated, homogenized liquid nutritional composition is packaged and the retort sterilized. In additional specific embodiments employing retort packaging, a packaged nutritional composition is retort heated at a temperature of at least about 250° F. (124° C.) for at least 5 minutes, although the retort conditions can be adjusted dependent on product properties, including product viscosity.

The specific method steps for heat treatments to obtain sterilization may be carried out in various ways other than those described above, without departing from the spirit and scope of the present invention. Regardless of the specific heat treatment process which is employed, and/or packaging which is employed, the inventive methods result in a storage stable nutritional composition in which the water-insoluble flavonoid remains suspended.

Specifically, the thus formed heat-treated liquid nutritional composition has a pH of from about 6 to about 7.5, more specifically, from about 6.5 to about 7.2, and comprises a water-insoluble plant flavonoid and protein. Both after the heat treatment and after storage of the heat-treated nutritional composition, the water-insoluble flavonoid remains suspended throughout the liquid composition. Specifically, at least about 75 wt %, more specifically at least about 80 wt %, at least about 85 wt %, or at least about 90 wt %, of the water-insoluble plant flavonoid remains suspended throughout the liquid nutritional composition, both after heat treatment and packaging, and after two months of storage at room temperature. In more specific embodiments, at least about 75 wt %, more specifically at least about 80 wt %, at least about 85 wt %, or at least about 90 wt %, of the water-insoluble plant flavonoid remains suspended throughout the liquid nutritional composition after five months of storage at room temperature. In more specific embodiments, at least about 75 wt %, more specifically at least about 80 wt %, at least about 85 wt %, or at least about 90 wt %, of the water-insoluble plant flavonoid remains suspended throughout the liquid nutritional composition after eight months of storage at room temperature. In further embodiments, at least about 75 wt %, more specifically at least about 80 wt %, at least about 85 wt %, or at least about 90 wt %, of the water-insoluble plant flavonoid remains suspended throughout the liquid nutritional composition after fifteen months of storage at room temperature. That the flavonoid remains suspended throughout the liquid nutritional composition is evidenced by the lack of any visual sedimentation in the bottles in which the composition is packaged. The compositions also substantially maintain original coloring, providing another visual indicator of good storage stability.

As described, it is believed that the high pressure homogenization increases association of the water-insoluble flavonoid with the protein in the liquid nutritional composition, whereby the flavonoid is maintained in suspension in the liquid composition. In a specific embodiment, the increased association is evidenced by the relative amounts of flavonoid and protein in resulting fractions when a liquid nutritional composition according to the invention is subjected to fractionation, for example, by centrifugation at 30,000×g, 3 hours. The water-insoluble plant flavonoid and protein appear to be at substantially the same weight and molar ratios in each resulting fraction, and these ratios are significantly higher than in a conventional liquid nutritional composition which is not formed with a homogenized flavonoid-containing formulation. This is particularly demonstrated in Example 2. Accordingly, in specific embodiments of the liquid nutritional compositions, upon centrifugation at 30,000×g, 3 hours, the water-insoluble plant flavonoid and protein in each resulting fraction are in a molar ratio of at least about 1.4:1, at least about 1.5:1, at least about 1.6:1, or at least about 1.7:1.

The nutritional compositions may further comprise one or more additional components that may modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

Additionally, the nutritional composition may further include vitamins or related nutrients, non-limiting examples of which include vitamin A, vitamin B12, vitamin C, vitamin D, vitamin K, thiamine, riboflavin, pyridoxine, niacin, folic acid, pantothenic acid, biotin, choline, inositol, salts and derivatives thereof, and combinations thereof. Water soluble vitamins may be added in the form of a water-soluble vitamin (WSV) premix and/or oil-soluble vitamins may be added in one or more oil carriers as desired.

In a specific embodiment, the liquid nutritional compositions contain hesperidin and/or hesperetin, and vitamin B12. While vitamin B12 is often degraded by flavonoids that are strong antioxidants such as the water-soluble green tea flavonoids, hesperidin and hesperetin have more moderate antioxidant activity, substantially comparable to that of ascorbic acid, and therefore do not significantly degrade vitamin B12. Nonetheless, because ascorbic acid is often included in nutritional compositions as well, to avoid excessive antioxidant activity, the concentration of flavonoid and any ascorbic acid contained in the liquid nutritional composition should not be greater than 5 mM. In specific embodiments, the liquid nutritional compositions comprise from about 1 to about 20 μg/L vitamin B12. In additional embodiments, the liquid nutritional compositions containing hesperidin and/or hesperetin, and vitamin B12 exhibit improved stability of the vitamin B12, for example, wherein after two months of storage at room temperature, at least about 75 wt %, at least about 80 wt %, at least about 85 wt %, or at least about 90 wt % of the vitamin B12 originally included in the liquid nutritional composition remains in the liquid nutritional composition.

In additional embodiments, the nutritional composition may further include one or more minerals, non-limiting examples of which include calcium, phosphorus, magnesium, zinc, manganese, sodium, potassium, molybdenum, chromium, chloride, and combinations thereof, for example, as citrates, phosphates, carbonates, sulfates, chlorides, or the like, or in other forms as known in the art.

In additional embodiments, the nutritional composition may further include one or more probiotics. The term “probiotic” as used herein refers to a microorganism such as a bacteria or yeast that survives the digestive process to confer a health benefit to the subject. Examples of probiotics that can be included in the nutritional compositions, either alone or in combination, include, but are not limited to, Bifidobacterium (B.), such as B. breve, B. infantis, B. lactis, B. bifidum, B. longum, and B. animalis, Lactobacillus (L.), such as L. rhamnosus, L. acidophilus, L. fermentum, and L. reuteri, Streptococcus thermophilus, Akkermansia, Bacteroides, Enterococcus, Eubacterium, Fecalibacterium, Roseburia, and/or Saccharomyces. The following Examples demonstrate various aspects of the invention.

### EXAMPLES

The examples illustrate specific aspects of the invention, are provided solely for the purpose of illustration, and are not to be construed as limiting of the general inventive concepts, as many variations thereof are possible without departing from the spirit and scope of the general inventive concepts.

### Example 1

In this example, two liquid nutritional compositions were prepared, each containing 500 mg hesperidin, supplied as Cardiose® from HealthTech BioActives (Murcia, Spain). The compositions had a pH of from about 6.5 to 7.2 and comprised milk protein concentrate and soy protein isolate in a weight ratio of about 65:35 as the protein sources, corn maltodextrin and sugar as the carbohydrate sources, and canola and corn oils as fat sources, providing 9 grams of protein, 33 grams of carbohydrate and 6 grams fat per 237 ml serving.

For the comparative composition, the hesperidin was added to the formulation comprising protein, fat and carbohydrate, and the composition was then subjected to UHT heat treatment and emulsification at 900-1000 psi. Water soluble vitamins and flavors were added, the total solids were adjusted, and the resulting nutritional composition was packaged and retort sterilized.

For the inventive composition, the hesperidin was added to the formulation comprising protein, fat and carbohydrate, and the composition was then subjected to UHT heat treatment and emulsification at 900-1000 psi, followed by a 2-stage homogenization process, with the first stage at 3000 psi and the second stage at 500 psi. After homogenization, water soluble vitamins and flavors were added and the total solids were adjusted. The resulting nutritional composition was packaged and retort sterilized.

The compositions were stored at room temperature for eight months and evaluated for suspended hesperidin content in the liquid products at two and eight months using HPLC as described in Example 2. At two months, the suspended hesperidin in the comparative composition (without high pressure homogenization) was 64.6 wt % of the hesperidin originally added to the formulation. The suspended hesperidin in the inventive composition (with high pressure homogenization) was 94.7 wt % of the hesperidin originally added to the formulation. At eight months, the suspended hesperidin in the comparative composition (without high pressure homogenization) was 43.2 wt % of the hesperidin originally added to the formulation. The suspended hesperidin in the inventive composition (with high pressure homogenization) was 93.6 wt % of the hesperidin originally added to the formulation.

The high pressure homogenization step provided a significant improvement in maintaining hesperidin in suspension in the liquid nutritional composition over the storage period.

### Example 2

In this example, comparative and inventive compositions prepared as described in Example 1 were subjected to fractionation analysis. The samples were subjected to a high speed centrifugation (30,000×g, 3 h) and the protein and hesperidin were analyzed in the collected phases to determine if hesperidin was associating with specific macronutrients in the product matrix that enabled it to remain in a stable colloidal suspension. Reverse phase HPLC analysis was used to quantify the water insoluble flavonoid (hesperidin) and protein in each fractionated product using the following methods:

HPLC Analytical Method for Hesperidin:
Column: YMC ODS-AQ, 4.6×250 mm, 120A, 5 um, Waters #AQ12S052546WT
Eluant A: 900 mL 0.02M KH2PO4, 100 mL Acetonitrile, pH 3.1
Eluant B: 200 mL Water, 800 mL Acetonitrile
Flow Rate: 0.5 mL/minute
Temperature: 20 C
Detection: UV/Vis at 280 nm
Injection: 5 uL
Run Time: 40 minutes

HPLA Analytical Method for Protein:
Column: Shodex Protein KW-803, 300×8 mm, Waters P/N WAT035946
Mobile Phase: 600 mL Milli-Q Plus water, 400 mL acetonitrile, 0.5 mL trifluoroacetic acid
Flow Rate: 0.3 mL/minute
Temperature: 20° C.
Detection: UV at 280 nm
Injection: 5 μL
Run Time: 53 minutes

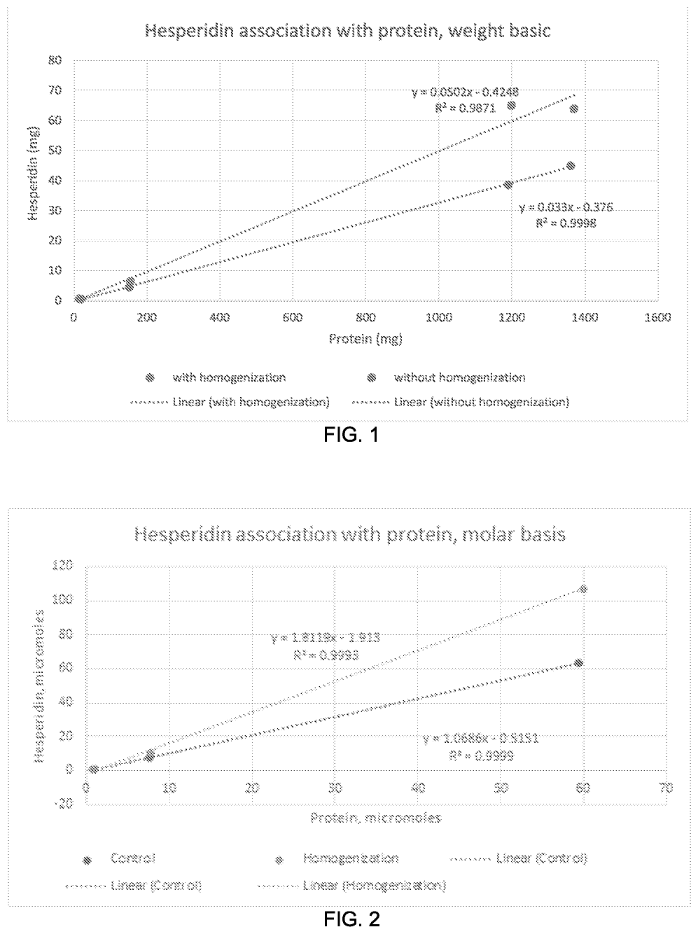

Table 1 and FIG. 1 show the fractionation of hesperidin and protein in each fractionation product of the comparative and inventive hesperidin-containing compositions, respectively, on a weight basis. Hesperidin showed preferential association with proteins in the matrix in each fractionation product:

TABLE 1

| Sample | Fraction | Protein (mg) | Hesperidin (mg) |
|---|---|---|---|
| Inventive - | Cream | 15.4 | 0.58 |
| Homogenization | Serum | 155.3 | 6.38 |
| | Pellet | 1199 | 65.01 |
| Comparative - | Cream | 19.6 | 0.49 |
| No Homogenization | Serum | 151.8 | 4.45 |
| | Pellet | 1189.9 | 38.53 |

Within each fraction, levels of hesperidin were found to correlate with the protein levels. Even when high centrifugal (30,000×g) force was exerted on the liquid formulation, hesperidin levels correlated with protein content in the cream, serum and pellet fractions. FIG. 1 shows the results graphically. Comparing the slopes of the curves in FIG. 1, homogenization enhanced the association between hesperidin and protein (0.0502/0.033) by 1.52 times. Furthermore, the mean apparent ratio between hesperidin to protein was significantly higher for the composition prepared with high pressure homogenization (0.044 (w/w)) compared with the control (0.029 (w/w)), further verifying the enhanced protein/hesperidin association. Without intending to be bound by theory, it is believed that this increased association between hesperidin and protein enhances the stability of hesperidin in the nutritional composition colloidal suspension. This association appears to be very stable since hesperidin was maintained in suspension even after eight months of shelf life testing. Because hesperidin is a water insoluble molecule, it was surprising that it was not concentrated in the cream fraction, the most lipid-rich fraction, but rather distributed with the protein fraction.

FIG. 2 shows the fractionation of hesperidin and protein in each fractionation product of the homogenized and non-homogenized hesperidin-containing compositions, respectively, on a molar basis. The slope of the comparative composition (1.0686) suggests a hesperidin/protein molar ratio of approximately 1:1. By contrast, the slope of the inventive composition (1.8119) is significantly higher than 1, indicating a substantial enhancement of the hesperidin/protein association by the homogenization.

While the present invention has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and methods, or illustrative examples shown and described. Accordingly, departures may be made from such details without departing from the spirit or scope of the general inventive concept.

1. A method of forming a heat-treated liquid nutritional composition having a pH of from about 6 to about 7.5, or from about 6.5 to about 7.2, and comprising a water-insoluble plant flavonoid, the method comprising
providing an aqueous liquid nutritional composition having a pH of from about 6 to about 7.5, or from about 6.5 to about 7.2, and comprising protein, fat, carbohydrate, and water-insoluble plant flavonoid,
homogenizing the liquid nutritional composition at a pressure of at least 2000 psi, or more specifically, from 2000 to about 5000 psi, and
heat treating the liquid nutritional composition.

2. The method of claim 1, wherein the liquid nutritional composition comprises from about 50 to about 800 mg, from about 50 to about 600 mg, or from about 50 to about 300 mg water-insoluble plant flavonoid per 237 ml of the liquid nutritional composition.

3. The method of claim 1, wherein the liquid nutritional composition comprises a weight ratio of protein to flavonoid of from about 5 to about 100, from about 10 to about 50, or from about 15 to about 25.

4. The method of claim 1, wherein the water-insoluble plant flavonoid comprises a citrus flavonoid, or the water-insoluble plant flavonoid comprises at least one of hesperidin and hesperetin.

5. The method of claim 1, wherein the water-insoluble plant flavonoid comprises resveratrol.

6. The method of claim 1, wherein the protein comprises milk protein, plant protein, or a combination thereof.

7. The method of claim 1, wherein the homogenizing step comprises at least one stage at a pressure of from about 2500 to about 5000 psi.

8. The method of claim 7, wherein the homogenizing step comprises a two-stage homogenization with a second stage at a pressure of from about 250 to about 1000 psi.

9. The method of claim 1, wherein the heat treatment provides sterilization sufficient to maintain product stability of the liquid nutritional composition over a shelf-life of at least about 12 months.

10. The method of claim 1, wherein the heat treatment is a high temperature, short time heat treatment at a temperature of from about 158° F. to about 176° F. (from about 70° C. to about 80° C.) for 10 to 25 seconds, or an ultra-high temperature (UHT) heat treatment at a temperature of from about 280° F. to about 305° F. (from about 138° C. to about 152° C.) for at least about 5 seconds.

11. The method of claim 1, wherein at least about 75 wt %, at least about 80 wt %, at least about 85 wt %, or at least about 90 wt % of the water-insoluble plant flavonoid remains suspended throughout the homogenized and heat treated liquid nutritional composition after two months, or more specifically, after eight months, of storage at room temperature.

12. A heat-treated liquid nutritional composition having a pH of from about 6 to about 7.5, or from about 6.5 to about 7.2, comprising a water-insoluble plant flavonoid, protein, fat and carbohydrate, wherein at least about 75 wt %, at least about 80 wt %, at least about 85 wt %, or at least about 90 wt % of the water-insoluble plant flavonoid remains suspended throughout the liquid nutritional composition after two months, or more specifically, after eight months, of storage at room temperature.

13. The liquid nutritional composition of claim 12, comprising from about 50 to about 800 mg, from about 50 to about 600 mg, or from about 50 to about 300 mg water-insoluble plant flavonoid per 237 ml of the liquid nutritional composition.

14. The liquid nutritional composition of claim 12, wherein the liquid nutritional composition comprises a weight ratio of protein to flavonoid of from about 5 to about 100, from about 10 to about 50, or from about 15 to about 25.

15. The liquid nutritional composition of claim 12, wherein the water-insoluble plant flavonoid comprises a citrus flavonoid, or the water-insoluble plant flavonoid comprises at least one of hesperidin and hesperetin.

16. The liquid nutritional composition of claim 12, wherein the water-insoluble plant flavonoid comprises resveratrol.

17. The liquid nutritional composition of claim 12, wherein the protein comprises milk protein, plant protein or a combination thereof.

18. The liquid nutritional composition of claim 12, wherein upon centrifugation at 30,000×g, 3 hours, the water-insoluble plant flavonoid and protein in each resulting fraction are in a molar ratio of at least about 1.4:1, at least about 1.5:1, at least about 1.6:1, or at least about 1.7:1.

19. The liquid nutritional composition of claim 12, further comprising about 1 to about 20 μg/L vitamin B12.

20. (canceled)

21. The liquid nutritional composition of claim 19, wherein after two months of storage at room temperature, at least about 75 wt %, at least about 80 wt %, at least about 85 wt %, or at least about 90 wt % of the vitamin B12 originally included in the liquid nutritional composition remains in the liquid nutritional composition.

22. The liquid nutritional composition of claim 19, wherein the concentration of the water-insoluble plant flavonoid and any ascorbic acid contained in the liquid nutritional composition is not greater than 5 mM.

23. The liquid nutritional composition of claim 12, comprising a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition.
